(19) United States
(12) Patent Application Publication
(10) Pub. No.: US 2005/0124592 A1
(43) Pub. Date: Jun. 9, 2005

(54) Tibolone in the treatment of complaints associated with the administration of drugs which prevent the synthesis of endogenous estrogen

(76) Inventors: Helenius Jan Kloosterboer, Oss (NL); Nigel Bundred, Manchester (GB)

Correspondence Address: AKZO NOBEL PHARMA PATENT DEPARTMENT, PO BOX 318, MILLSBORO, DE, 19966, US

(21) Appl. No.: 10/502,444

(22) PCT Filed: Jan. 16, 2003
(86) PCT No.: PCT/EP03/00373

(30) Foreign Application Priority Data
Jan. 22, 2002 (EP) 02075235.8

(51) Int. Cl. A61K 31/56; A61K 31/4178; A61K 31/137; A61K 31/165

(52) U.S. Cl. 514/169; 514/649; 514/397; 514/617

(57) ABSTRACT

Disclosed is a treatment of estrogen-deficiency related complaints in females that exhibit these complaints while they are on treatment with a drug which prevents the synthesis of endogenous estrogen. Such drugs are, e.g., anti-cancer drugs such as aromatase inhibitors. The invention resides in the use of tibolone, which has an unexpectedly beneficial working in this particular patient group in that it does not stimulate breast, while preventing bone loss and relieving climacteric complaints in a patient group in which this is more difficult than in any other group due to the nature of the concomitant cancer treatment (no circulating estrogen making for a higher severity of the complaints, the lack of effect on the estrogen receptor making for an increased risk associated with estrogenic breast stimulation once estrogen-like compounds are administered.

The invention pertains to the treatment of estrogen-deficiency related complaints in females that exhibit these complaints while they are on treatment with a drug which prevents the synthesis endogenous (active) estrogens, notably estradiol. Such drugs are, e.g., anti-cancer drugs such as aromatase inhibitors, 17β hydroxy steroid dehydrogenase inhibitors, sulfatase inhibitors.

Estrogen-deficiency related complaints, such as climacteric complaints and bone loss, are well-known as symptoms in (post)menopausal women. For these ilinessess and symptoms, various treatments exist, such as estradiol suppletion, combination of estrogens and progestagens, and other drugs.

Another patient group comprises females which—whether before or after the natural menopause—due to some treatment or surgery exhibit complaints which are estrogen-deficiency related. Well known are the effects of the administration of a partial estrogen receptor antagonist such as tamoxifen, or selective estrogen receptor modulators such as raloxifene. To the extent that the female patients exhibit the above-indicated complaints, it were desirable if a suitable treatment was available. The problem, however, with regular drugs for the treatment of estrogen-deficiency related complaints is that they cannot be used in patients which have, or have had, breast cancer or are known to have a risk for breast cancer. The reason is that the typical drugs used for estrogen-supplementation will increase the recurrence of, or even cause, breast tumors. In fact, it is one of known effects of estrogens and estrogen-like therapies that they stimulate breast.

A special population of female patients having the above-indicated symptoms comprises those that are subject to treatment with drugs which act on the metabolic pathway which leads to the synthesis of endogenous estrogens rather than at the level of the estrogen receptors. These drugs include aromatase inhibitors and 17β, hydroxy steroid dehydrogenase inhibitors, sulfatase inhibitors. Other than in natural (post)menopausal women—who still have circulating estrogen formed from precursors produced by the adrenals—or women that are on treatment with drugs acting at receptor level—who have circulating estrogen but see its action competed by estrogen receptor antagonism, females on treatment with aromatase inhibitors or other drugs that prevent estradiol from being synthesized, have lack of circulating estrogen. While this is clearly an advanced treatment, further reducing the risk of estrogen-dependent tumors occurring, the female patients thus treated will run an even higher chance and/or higher severity of estrogen-deificiency related complaints.

In treating these complaints with classical hormone replacement therapy, the risk of estrogen-like treatment on the stimulation of growth of tumors is even higher than in patients treated with receptor antagonists, since any supplemented estrogen will not be antagonized, and thus exert its full effect. Moreover, due to the stringency of the treatment (preferably a total or near complete prevention of estrogen exposure, and notably estradiol synthesis), it is an even greater challenge to treat the complaints than in the case of either natural (post)menopausal women, or those that—while on antagonist or SERM treatment (selective estrogen receptor modulators)—still have circulating estrogen.

According to the invention, one drug has been found which presents a solution to the above dilemma, viz. tibolone. This is an unexpected finding, not only because of the inherent difficulty in finding any treatment at all in the above special population, but also because tibolone itself hardly has an estrogenic activity, and is metabolized to compounds which have an approximately fifty-fold lower estrogenic receptor activity than estradiol. That particularly this drug works in the treatment of complaints related to a (near) total lack of circulating estrogen, is unprecedented.

The compound tibolone, (7α,17α)-17-hydroxy-7-methyl-19-nor-17-pregn-5(10)-en-20-yn-3-one, is known as a tissue-specific and effective agent that can be used in hormone replacement therapy (HRT) in (post)menopausal women, for the treatment of menopausal and postmenopausal disorders, including climacteric complaints, vasomotor symptoms, osteoporosis, and vaginal atrophy. See, int.al., U.S. Pat. No. 5,037,817 and WO 98/47517

Tibolone is a synthetic compound, which shows weak estrogenic, androgenic and progestagenic activities to estrogen, progesterone, and androgen receptors. Previous studies have shown favorable effects on bone, the vagina, the cardiovascular system, climacteric symptoms, mood, and libido without detrimental estrogen-like stimulation of the breast and endometrium (Kloosterboer, 2001; Kloosterboer et al., 2000; Pain Research and Nuffield Department of Anaesthetics, 1999; Tang et al., 1993). Studies have indicated that tibolone increases BMD relative to baseline or placebo over periods ranging from six months to three years (Pain Research and Nuffield Department of Anaesthetics, 1999).

Tibolone, at any rate prior to this invention, is subject to a warning for use in cancer-endangered patients. Tibolone is known from EP 613687 in the prevention or treatment of tumors. It should be noted that this relates to a different medical indication than that according to the invention.

The use of tibolone in the special population discussed above has not been disclosed in the art, nor can its favourable and safe activity be derived therefrom.

The compound of the invention may be administered enterally or parenterally, and for humans in a daily dosage of 0.003-3.0 mg per kg body weight; preferably a daily dosage of 0.03-0.4 mg per kg body weight is administered. More preferably, the invention can be carried out by providing tibolone in daily dosage amounts of from 0.2 to 5 mg, preferably 0.4 to 2.5 mg and more preferably fixed dosages of 1.25 or 2.5mg.

Mixed with pharmaceutically suitable auxiliaries, e.g. as described in the standard reference, Gennaro et al., Remington's Pharmaceutical Sciences, (18th ed., Mack Publishing Company, 1990, see especially Part 8: Pharmaceutical Preparations and Their Manufacture) the compound may be compressed into solid dosage units, such as pills, tablets, or be processed into capsules or suppositories. By means of pharmaceutically suitable liquids the compound can also be applied as an injection preparation in the form of a solution, suspension, emulsion, or as a spray, e.g. a nasal spray. For making dosage units, e.g. tablets, the use of conventional additives such as fillers, colorants, polymeric binders and the like is contemplated. In general, any pharmaceutically acceptable additive which does not interfere with the function of the active compound can be used.

Suitable carriers with which the compositions can be administered include lactose, starch, cellulose derivatives and the like, or mixtures thereof, used in suitable amounts.

Thus an Example of a tablet of tibolone has the following composition:

| | |
|---|---|
| tibolone | 2.5 mg |
| starch | 10 mg |
| ascorbyl palmitate | 0.2 mg |
| magnesium stearate | 0.5 mg |
| lactose | to make up to 100 mg |

And is made from base granules prepared by mixing the lactose with a portion of the starch. The remainder of the starch was mixed to a slurry with water and added to the mixture. The whole was granulated and dried. These base granules were mixed with ascorbyl palmitate and compound I, sieved, finely mixed with magnesium stearate and then tabletted.

The patient population to which the present invention applies will generally be on treatment with one or more of the following drugs aminogluthethimide, anastrozole, letrozole, exemestane, formestane or other inhibitors or inactivators of aromatase, or of other enzymes which affect estradiol synthesis such as of sulfatase of 17β-hydroxysteroid dehydrogenase. These drugs will generally be used in their regular therapeutically effective doses. E.g., anastrozole will typically be used in 1 or 10 mg/day, letrozole in 2.5 mg/day, formestane e.g. 250 or 500 mg i.m. fortnightly. The invention is not limited to the above compounds and dosages, the essence being in the type of treatment: the prevention of the synthesis of active estrogens, notably the synthesis of estradiol.

In summary the invention is a method of treatment of estrogen-deficiency related complaints in females that exhibit these complaints while they are on treatment with a drug which prevents the synthesis of active estrogens. Such drugs are, e.g., anti-cancer drugs such as aromatase inhibitors and inactivators. The invention resides in the use of tibolone, which has an unexpectedly beneficial working in this particular patient group in that it does not stimulate breast, while preventing bone loss and relieving climacteric complaints in a patient group in which this is more difficult than in any other group due to the nature of the concomittant cancer treatment (no circulating estrogen making for a higher severity of the complaints, the lack of effect on the estrogen receptor making for an increased risk associated with estrogenic breast stimulation once estrogen-like compounds are administered.

1. (canceled)

2. (canceled)

3. (canceled)

4. (canceled)

5. (canceled)

6. (canceled)

7. A method of treatment of estrogen-deficiency related complaints in female patients that exhibit these complaints while the female patients are on treatment with a drug which prevents the synthesis of endogenous estrogen, comprising:
administering to said female patients of an effective amount of tibolone.

8. The method of claim 7, wherein the estrogen-deficiency related complaints are climacteric complaints.

9. The method of claim 7, wherein the estrogen-deficiency related complaints are bone loss.

10. The method of claim 7, wherein the drug which prevents the synthesis of endogenous estrogen is an aromatase inhibitor.

12. The method of claim 7, wherein tibolone is administered in a daily dosage of 0.4 to 2.5 mg.

13. The method of claim 7, wherein the aromatase inhibitor is selected from the group consisting of aminogluthethimide, anastrozole, letrozole, exemestane, and formestane.
